(19) United States
(12) Patent Application Publication
(10) Pub. No.: US 2017/0281400 A1
(43) Pub. Date: Oct. 5, 2017

(54) INSERT FOR HUMANS AND ANIMALS AND THE PROCESS FOR PRODUCING THE SAME

(71) Applicants: CABALLUS CARNIOLUS & AMSTERDAMUS, VETERINARSKO TEHNOLOSKI CENTER IN TRGOVINA, UVOZ-IZVOZ D.O.O., Kranj (SI); VETERINARY RESEARCH CENTRE LLC, Bridgeton, NJ (US)

(72) Inventors: Tilen KLEVISAR, Kranj (SI); Almir KARABEGOVIC, Prijedorska (SI)

(21) Appl. No.: 15/507,682

(22) PCT Filed: Dec. 24, 2014
(86) PCT No.: PCT/SI2014/000083

§ 371 Date: Feb. 28, 2017

(30) Foreign Application Priority Data
Aug. 28, 2014 (SI) P-201400292

(51) Int. Cl. A61F 6/14

(52) U.S. Cl. CPC A61F 6/142; A61F 6/06

(57) ABSTRACT

The object of the invention belongs to the field of contraceptive devices, more precisely to the field of inserts for humans and animals intended for contraception.

The essence of the insert according to the invention is that the intra-vaginal or uterine insert is shaped as a letter V, wherein the insert is made of into a spiral coiled stainless steel, which is galvanized with one or more different metals selected in the group consisting of copper, silver and copper in combination with gold. The insert has two modes of function, contraceptive and healing, wherein contraceptive function is more important for human females, while the healing effect has higher importance for female animals.

Described insert for humans and animals according to the invention allows safe use. In addition, the insert with added gold has healing properties. At the same time, adjustment of dimensions depending on human or animal needs is possible, so that the insert fits perfectly, and therefore has optimal activity. Furthermore, insert insertion can be carried out according to standard and already known methods.

### FIELD OF THE INVENTION

The object of the invention is an insert for humans and animals, which belongs to the field of contraceptive devices for barrier prevention of conception, more precisely to the field of uterine inserts for humans and animals.

### Technical Problem

The technical problem, which is solved by the present invention, is the constructional design of an insert for female humans and female animals, which will enable reliable contraception, and will allow human and animal pregnancy after intentionally discontinued use. The main purpose of this invention is to design an insert, which will enable reliable barrier prevention of conception with reduced negative impacts on the user. The insert should minimize undesirable health effects, which usually occur due to the choice of materials for insert manufacture.

The design of the insert according to the present invention should allow easy insertion and placement, effective contraception and safe use, which includes normal hormonal balance and pregnancy after discontinued use of the insert. At the same time, the design of the insert should also allow use as a healing insert, as it should have positive effects in case of infections and other problems connected with reproductive organs.

### STATE OF THE ART

Little devices, which absorb and emit sexual hormones, and in this way control conception, are often used for animal contraception, mainly for pets. Intra-vaginal or uterine inserts can also be used for this purpose.

Many methods and devices for human female contraception are known. In addition to contraceptive medicines, uterine inserts are most commonly used among devices, which physically prevent conception.

Intra-vaginal inserts, which are used as contraceptive means, can be made in different shapes, including:
heart shape: disclosed in U.S. Pat. No. 3,516,403 in RU2106837;
shape of a ball composed from three rings: disclosed in patent CN201987726;
the shape of a cylinder with three circular cylinders: described in patent CN1919161;
shape of trefoil: disclosed in patent JP2006314809;
spiral shape: described in patent RU1811825; and
shape of a letter T or a letter V.

Intra-vaginal inserts in the shape of a letter V for animals have been described in patent documents JP1172, EP0803237, and BG99711, and for humans in patent documents CN2661137 in CN102908224.

Patent documents JP1172 in EP0803237 disclose a uterine insert for female dogs, which has a body and two branches in the shape of a letter V, wherein an opening is located on the bottom of the insert so that a nylon string for easier insert removal can be attached, while there is a short flat surface on both ends of branches on the upper side of the insert. The body and both branches of the insert are wrapped with copper filaments.

Patent document BG99711 also discloses a uterine insert for female dogs in the shape of a letter V, which is composed from two hollow branches between which there is a 45 degree angle, wherein one branch ends with a copper plate, while the other ends with a silver plate. Both branches are filled with hormone progesterone, which chemically prevents animal pregnancy.

Patent application CN2661137 discloses a contraceptive device for female humans in the shape of a letter V, which is made of a memory alloy wire and is coated with a medical grade silicon rubber, and has on each end a circular ending.

Patent document CN102908224 discloses a uterine insert from a medically acceptable memory alloy wire, which has an integrated spiral made of a copper spiral, wherein the wire is wrapped with a silicon rubber. The insert is shaped as a letter V on the top, there is an approximately rectangular part in the middle, while on the bottom the insert is shaped as a circle.

None of the above mentioned solutions includes an intra-vaginal insert, which would be made of into a spiral coiled stainless steel, which is galvanized with different metals, as is the case in the present invention. In addition, none of the known solutions describes a possible healing effect of inserts.

### Solution of the Technical Problem

The essence of the insert according to the invention is that the intra-vaginal or uterine insert is shaped as a letter V, wherein the insert is made of into a spiral coiled stainless steel, which is galvanized with one or more different metals selected in the group consisting of copper, silver and copper in combination with gold, and the spiral radium and branch length depends on the recipient of the insert.

Insert for humans and animals will be described in further detail with a detailed description and a FIGURE, which shows:

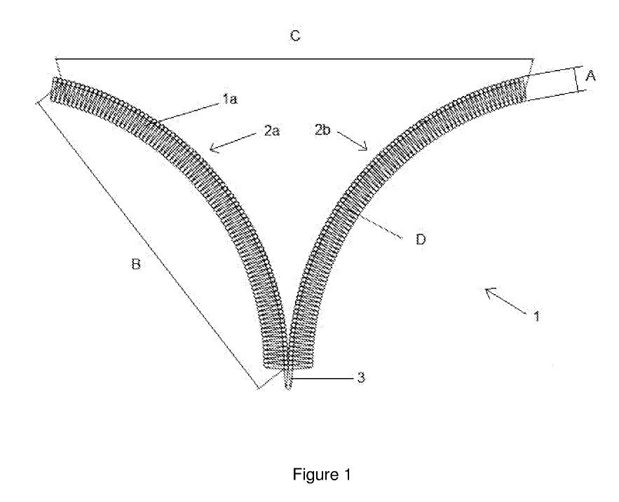

FIG. 1 Insert for humans and animals according to the invention

Insert 1 is basically a spiral 1a made from a metal wire, which is divided into two branches 2a and 2b, wherein the bottom part 3 is ring-shaped and represents the middle part of the spiral 1. The spiral 1 is from the ring part 3 curved upwards so that branches 2a and 2b are curved as arcs pointing to opposite sides, thereby forming a letter V. The symmetrical branches 2a and 2b are connected with the ring part 3, which allows attachment of a string or any other means for easier insert removal. The spiral 1 is made of stainless steel, which is coiled into a thick spiral with a diameter of 1.3 mm. The arc length B of branches 2a and 2b may be between 20 and 30 mm, while the angle C between both branches may be between 76° and 77°. Radius D of each branch may be between 14.0 and 22.5 mm. The insert weight is between 0.3 and 0.5 g. These dimensions enable size adjustment depending on the animal or human female size, as each insert must fit perfectly to the uterus in order to function properly.

The process for manufacturing of the insert for humans and animals according to the invention begins with preparation of into a spiral coiled stainless steel (1.4301), wherein the spiral diameter is 1.3 mm, the diameter of wire is 0.299 mm, and on every 10 mm of the spiral there are 33 coils. Stainless steel is then galvanized with different metals selected in the group consisting of copper, copper combined with gold, and silver. Materials used in the insert manufacturing process have a quality degree acceptable for medical use. Regardless of the selected metal, the thickness of the galvanization deposit is from 10 to 30 μm, preferably 20 μm, wherein the weight of added gold, in case of copper combined with gold, is 2 grams.

The insert according to the invention has two modes of function, contraceptive and healing, wherein contraceptive function is more important for human females, while the healing effect has higher importance for female animals.

Use of copper in contraceptive inserts and devices is common, as copper is toxic for semen. However, copper is also toxic for eggs and uterine cells, therefore its presence should be limited or alleviated. The combination of copper and gold is appropriate for this purpose. Gold has a positive effect on cells and consequently limits or nullifies negative effects of copper on uterine cells.

The insert can have healing effects, because gold, due to its elementary properties, positively affects cells and therefore has a healing effect on different problems with reproductive organs. For example, the insert can limit and heal infections of reproductive organs, polyps of reproductive organs or tumours of reproductive organs.

### EMBODIMENT I

Contraceptive insert for humans and animals according to the embodiment I is made of into a spiral coiled stainless steel (1.4301), wherein the diameter of the spiral is 1.3 mm, the diameter of the wire is 0.299 mm, and on every 10 mm of the spiral there are 33 coils. Stainless steel is galvanized with copper. The thickness of the galvanization deposit is 10 to 30 μm, preferably 20 μm. From described stainless steel an insert in the shape of a letter V is made with the following dimensions:
arc length: 20,003 mm;
angle: 76.6°;
radius: 14,964 mm; and
weight: 0.31 g.

### EMBODIMENT II

Contraceptive insert for humans and animals according to the embodiment I is made of into a spiral coiled stainless steel (1.4301), wherein the diameter of the spiral is 1.3 mm, the diameter of the wire is 0.299 mm, and on every 10 mm of the spiral there are 33 coils. Stainless steel is galvanized with copper. The thickness of the galvanization deposit is 10 to 30 μm, preferably 20 μm. From described stainless steel an insert in the shape of a letter V is made with the following dimensions:
arc length: 24.89 mm;
angle: 76.5°;
radius: 18,642 mm; and
weight: 0.38 g.

### EMBODIMENT III

Contraceptive insert for humans and animals according to the embodiment I is made of into a spiral coiled stainless steel (1.4301), wherein the diameter of the spiral is 1.3 mm, the diameter of the wire is 0.299 mm, and on every 10 mm of the spiral there are 33 coils. Stainless steel is galvanized with copper. The thickness of the galvanization deposit is 10 to 30 μm, preferably 20 μm. From described stainless steel an insert in the shape of a letter V is made with the following dimensions:
arc length: 30 mm;
angle: 76.76°;
radius: 22,393 mm; and
weight: 0.45 g.

### EMBODIMENT IV

Contraceptive insert for humans and animals according to the embodiment I is made of into a spiral coiled stainless steel (1.4301), wherein the diameter of the spiral is 1.3 mm, the diameter of the wire is 0.299 mm, and on every 10 mm of the spiral there are 33 coils. Stainless steel is galvanized with copper combined with gold. The thickness of the galvanization deposit is 10 to 30 μm, preferably 20 μm. From described stainless steel an insert in the shape of a letter V is made with the following dimensions:
arc length: 20,003 mm;
angle: 76.6°;
radius: 14,964 mm; and
weight: 0.31 g.

### EMBODIMENT V

Contraceptive insert for humans and animals according to the embodiment I is made of into a spiral coiled stainless steel (1.4301), wherein the diameter of the spiral is 1.3 mm, the diameter of the wire is 0.299 mm, and on every 10 mm of the spiral there are 33 coils. Stainless steel is galvanized with copper combined with gold. The thickness of the galvanization deposit is 10 to 30 μm, preferably 20 μm. From described stainless steel an insert in the shape of a letter V is made with the following dimensions:
arc length: 24.89 mm;
angle: 76.5°;
radius: 18,642 mm; and
weight: 0.38 g.

### EMBODIMENT VI

Contraceptive insert for humans and animals according to the embodiment I is made of into a spiral coiled stainless steel (1.4301), wherein the diameter of the spiral is 1.3 mm, the diameter of the wire is 0.299 mm, and on every 10 mm of the spiral there are 33 coils. Stainless steel is galvanized with copper combined with gold. The thickness of the galvanization deposit is 10 to 30 μm, preferably 20 μm. From described stainless steel an insert in the shape of a letter V is made with the following dimensions:
arc length: 30 mm;
angle: 76.76°;
radius: 22,393 mm; and
weight: 0.45 g.

### EMBODIMENT VII

Contraceptive insert for humans and animals according to the embodiment I is made of into a spiral coiled stainless steel (1.4301), wherein the diameter of the spiral is 1.3 mm, the diameter of the wire is 0.299 mm, and on every 10 mm of the spiral there are 33 coils. Stainless steel is galvanized with silver. The thickness of the galvanization deposit is 10 to 30 μm, preferably 20 μm. From described stainless steel an insert in the shape of a letter V is made with the following dimensions:
arc length: 20,003 mm;
angle: 76.6°;
radius: 14,964 mm; and
weight: 0.31 g.

### EMBODIMENT VIII

Contraceptive insert for humans and animals according to the embodiment I is made of into a spiral coiled stainless steel (1.4301), wherein the diameter of the spiral is 1.3 mm, the diameter of the wire is 0.299 mm, and on every 10 mm of the spiral there are 33 coils. Stainless steel is galvanized with silver. The thickness of the galvanization deposit is 10 to 30 μm, preferably 20 μm. From described stainless steel an insert in the shape of a letter V is made with the following dimensions:
arc length: 24.89 mm;
angle: 76.5°;
radius: 18,642 mm; and
weight: 0.38 g.

### EMBODIMENT IX

Contraceptive insert for humans and animals according to the embodiment I is made of into a spiral coiled stainless steel (1.4301), wherein the diameter of the spiral is 1.3 mm, the diameter of the wire is 0.299 mm, and on every 10 mm of the spiral there are 33 coils. Stainless steel is galvanized with silver. The thickness of the galvanization deposit is 10 to 30 μm, preferably 20 μm. From described stainless steel an insert in the shape of a letter V is made with the following dimensions:
arc length: 30 mm;
angle: 76.76°;
radius: 22,393 mm; and
weight: 0.45 g.

Insert for humans and animals, according to the invention, is inserted according to any known method appropriate for insertion. Insert for humans and animals according to the invention can be easily maintained and changed, as these processes are the same as for all already known uterine inserts.

Described insert for humans and animals according to the invention allows safe use, as pregnancy is possible after intentionally discontinued use of the insert with the aim of conception. In addition, insert with added gold has healing properties especially for animals, as it limits infections of reproductive organs, as well as polyps and tumors of reproductive organs. At the same time, adjustment of dimensions depending on human or animal needs is enabled, so that the insert fits perfectly, and therefore has optimal activity. Furthermore, use of the insert for humans and animals according to the invention does not require any additional learning or special insertion apparatus, because the latter can be carried out according to standard and already known methods.

1. An insert for humans and animals comprising a V-shape, wherein:
the insert (1) comprises two curved branches (2a in 2b) in the shape of an arc,
on the bottom is a ring part (3), onto which a string (4) can be optionally attached,
the insert has a spiral diameter (A) of 1.3 mm, arc length (B) between 20 and 30 mm, angle (C) between both branches 76 and 77° and radius (D) between 14.0 and 22.5 mm;
and that the insert is made of into a spiral coiled stainless steel, which is galvanized with one or more metal selected from the group consisting of copper, silver and copper in combination with gold, and that the thickness of the galvanization deposit is from 10 to 30 μm, preferably 20 μm.

2. The insert of claim 1, characterized in that the stainless steel is galvanized with copper.

3. The insert of claim 1, characterized in that the stainless steel is galvanized with copper combined with gold.

4. The insert of claim 1, characterized in that the stainless steel is galvanized with silver.

5. The insert of claim 1 comprising the following dimensions:
arc length: 20,003 mm;
angle: 76.6°;
radius: 14,964 mm; and
weight: 0.31 g.

6. The insert of claim 1 comprising the following dimensions:
arc length: 24.89 mm;
angle: 76.5°;
radius: 18,642 mm; and
weight: 0.38 g.

7. The insert of claim 1 comprising the following dimensions:
arc length: 30 mm;
angle: 76.76°;
radius: 22,393 mm; and
weight: 0.45 g.

8. The insert of claim 1 for use in preventing pregnancy in women.

9. The insert of claim 1 for use in treating infections of uterus, polyps of reproductive organs and tumours of reproductive organs in female animals.
